(19) United States
(12) Patent Application Publication
(10) Pub. No.: US 2025/0161197 A1
(43) Pub. Date: May 22, 2025

(54) TEETH STAIN BLOCKING COMPOSITION AND METHOD OF USE

(71) Applicants: Ashley Beirne, Chicago, IL (US); Mariam Morienyane, London (GB)

(72) Inventors: Ashley Beirne, Chicago, IL (US); Mariam Morienyane, London (GB)

(21) Appl. No.: 18/841,667

(22) PCT Filed: Feb. 27, 2023
(86) PCT No.: PCT/US2023/063376

§ 371 Date: Aug. 26, 2024

Related U.S. Application Data
(60) Provisional application No. 63/268,555, filed on Feb. 25, 2022.

(51) Int. Cl. A61K 8/9789; A61K 8/19; A61K 8/34; A61K 8/67; A61K 8/92; A61K 8/9717; A61Q 11/00

(52) U.S. Cl. CPC A61K 8/9789; A61K 8/19; A61K 8/345; A61K 8/676; A61K 8/922; A61K 8/9717; A61Q 11/00

(57) ABSTRACT

An aqueous oral composition is provided to reduce, limit and/or prevent staining of the teeth and/or oral cavity. An aqueous oral composition comprises (A) an alcohol, (B) sodium bicarbonate, (C) an artificial sweetener, (D) one or more vitamins or vitamin-containing compositions, (E) grape seed extract, and (F) miswak powder.

### RELATED APPLICATIONS

This application claims priority to U.S. Provisional Application No. 63/268,555, filed on Feb. 25, 2022, the entire disclosure of which is incorporated herein by reference.

### TECHNICAL FIELD

This application is directed to a composition which, when applied to teeth, reduces or prevents staining.

### BACKGROUND

When consuming red wine, it often results in what is referred to as “red wine mouth,” or the coloring of one's mouth, including teeth, in a redder shade than normal. This short-term discoloration is not only a social nuisance, but it can also be damaging to the teeth. Indeed, continual short-term discoloration can eventually lead to long-term or permanent staining. Also, the acidity and pH level of certain beverages and food can damage tooth enamel, regardless of whether those beverages or foods stain teeth.

Existing products intended to reduce mouth and teeth staining, particularly when enjoying beverages, can be conspicuous to use and even change the taste of foods and beverages. For example, existing hydrogen peroxide wipes intended to remove staining after a meal have a chemical taste and are conspicuous to use. Another post-meal solution is a dissolvable tablet which loosens coloration and therefore reduces/removes staining. Such tablets, while convenient and often well-flavored, contain a significant amount of artificial ingredients and chemicals. Other products use a balm-type composition which needs to be reapplied frequently in order to prevent staining. While less conspicuous than a mouth wipe, balms inherently leave a residue on lips and teeth that can be uncomfortable and alter the taste of beverages and foods.

Thus, a need exists for an effective solution to teeth and mouth staining and damage from beverages and foods that avoids the drawbacks of existing solutions.

### SUMMARY

In an embodiment, the present disclosure provides an aqueous oral composition. In accordance with embodiments of the present disclosure, the aqueous oral composition comprises (A) an alcohol, (B) sodium bicarbonate, (C) an artificial sweetener, (D) one or more vitamins or vitamin-containing compositions, (E) grape seed extract, and (F) miswak powder.

In an embodiment, the alcohol is present in an amount from 5 wt % to 25 wt %. In another embodiment, the alcohol is selected from the group consisting of glycerin, sorbitol, and combinations thereof. In a further embodiment, the sodium bicarbonate is present in an amount from 1 wt % to 12 wt %. In still a further embodiment, the artificial sweetener is present in an amount from 1 wt % to 5 wt %. In another embodiment, the artificial sweetener is selected from the group consisting of xylitol, stevioside, sodium saccharin, and combinations thereof. In yet another embodiment, the one or more vitamins or vitamin-containing compositions is present in an amount from 0.1 wt % to 4 wt %. In a particular embodiment, the one or more vitamins or vitamin-containing compositions is selected from the group consisting of vitamin C, red algae gel, and combinations thereof. In an embodiment, the one or more vitamins or vitamin-containing compositions comprises vitamin C and red algae gel. In another embodiment, the vitamin C and red algae gel are independently present in an amount from 0.1 wt % to 2 wt %. In still another embodiment, the grape seed extract is present in an amount from 0.1 wt % to 1 wt %. In a further embodiment, the miswak powder is present in an amount from 0.1 wt % to 1 wt %.

In an embodiment, the aqueous oral composition further comprises an additional component selected from the group consisting of preservatives, solvents, emulsifiers, natural oils, surfactants and combinations thereof. In an embodiment, the aqueous oral composition further includes a preservative selected from the group consisting of benzyl alcohol, cetylpyrdium chloride, and combinations thereof. In another embodiment, the aqueous oral composition further includes a solvent/emulsifier which is triethylene glycol. In yet another embodiment, the aqueous oral composition further comprises a natural oil which is fractionated coconut oil. In a further embodiment, the aqueous oral composition comprises a surfactant comprising glycolipids.

In an embodiment, the aqueous oral composition is provided as a spray, rinse, or liquid to be applied with an applicator.

In an embodiment, a method of using an aqueous oral composition is disclosed. In accordance with embodiments of the present disclosure, the method comprises spraying the aqueous oral composition in an oral cavity. In a further embodiment, the step of spraying the aqueous oral composition in an oral cavity comprises spraying the aqueous oral composition on a user's teeth.

### DEFINITIONS AND TEST METHODS

Any reference to the Periodic Table of Elements is that as published by CRC Press, Inc., 1990-1991. Reference to a group of elements in this table is by the new notation for numbering groups.

For purposes of United States patent practice, the contents of any referenced patent, patent application or publication are incorporated by reference in their entirety (or its equivalent US version is so incorporated by reference) especially with respect to the disclosure of definitions (to the extent not inconsistent with any definitions specifically provided in this disclosure) and general knowledge in the art.

The numerical ranges disclosed herein include all values from, and including, the lower and upper value. For ranges containing explicit values (e.g., a range from 1, or 2, or 3 to 5, or 6, or 7), any subrange between any two explicit values is included (e.g., the range 1-7 above includes subranges 1 to 2; 2 to 6; 5 to 7; 3 to 7; 5 to 6; etc.).

Unless stated to the contrary, implicit from the context, or customary in the art, all parts and percentages are based on weight and all test methods are current as of the filing date of this disclosure.

“Blend” refers to a composition of two or more components. Such a blend may or may not be miscible. Such a blend may or may not be phase separated. Such a blend may or may not contain one or more domain configurations, as determined from transmission electron spectroscopy, light scattering, x-ray scattering, and any other method used to measure and/or identify domain configurations.

The term “composition” refers to a mixture of materials which comprise the composition, as well as reaction products and decomposition products formed from the materials of the composition.

The terms “comprising,” “including,” “having,” and their derivatives, are not intended to exclude the presence of any additional component, step or procedure, whether or not the same is specifically disclosed. In order to avoid any doubt, all compositions claimed through use of the term “comprising” may include any additional additive, adjuvant, or compound, whether polymeric or otherwise, unless stated to the contrary. In contrast, the term “consisting essentially of” excludes from the scope of any succeeding recitation any other component, step, or procedure, except those that are not essential to operability. The term “consisting of” excludes any component, step, or procedure not specifically delineated or listed. The term “or,” unless stated otherwise, refers to the listed members individually as well as in any combination. Use of the singular includes use of the plural and vice versa.

### DETAILED DESCRIPTION

The present disclosure provides an aqueous oral composition comprising (A) an alcohol, (B) sodium bicarbonate, (C) an artificial sweetener, (D) one or more vitamins or vitamin-containing compositions, (E) grape seed extract, and (F) miswak powder.

### A. Alcohol

The aqueous oral composition comprises (A) an alcohol. The alcohol is present in an amount from 5 wt %, or 10 wt %, or 15 wt % to 20 wt % or 25 wt %. In a further embodiment, the alcohol is present in an amount from 10 wt %, or 12 wt %, or 14 wt %, or 16 wt %, or 18 wt % to 20 wt %, or 22 wt %, or 24 wt %, or 25 wt %.

The alcohol acts as a solvent and bodying agent to provide the composition with a favorable mouth feel. Nonlimiting examples of suitable alcohols include glycerin, sorbitol, and combinations thereof. In an embodiment, the alcohol is glycerin.

### B. Sodium Bicarbonate

The aqueous oral composition comprises (B) sodium bicarbonate. The sodium bicarbonate is present in an amount from 1 wt %, or 2 wt %, or 3 wt %, or 4 wt %, or 5 wt %, or 6 wt % to 7 wt %, or 8 wt %, or 9 wt %, or 10 wt %, or 11 wt %, or 12 wt %. In a further embodiment, the sodium bicarbonate is present in an amount from 3 wt %, or 3.25 wt %, or 3.5 wt %, or 3.75 wt %, or 4 wt %, or 4.25 wt %, or 4.5 wt %, or 4.75 wt %, or 5 wt %, or 5.25 wt %, or 5.5 wt %, or 5.75 wt %, or 6 wt %, or 6.25 wt %, or 6.5 wt %, or 6.75 wt % to 7 wt %, or 7.25 wt %, or 7.5 wt %, or 7.75 wt %, or 8 wt %, or 8.25 wt %, or 8.5 wt %, or 8.75 wt %, or 9 wt %, or 9.5 wt %, or 10 wt %.

Sodium bicarbonate is a known teeth cleaning agent and has antiseptic properties.

### C. Artificial Sweetener

The aqueous oral composition comprises (C) an artificial sweetener. The artificial sweetener is present in an amount from 1 wt %, or 1.5 wt %, or 2 wt %, or 2.5 wt % to 3 wt %, or 3.5 wt %, or 4 wt %, or 4.5 wt %, or 5 wt %. In a further embodiment, the artificial sweetener is present in an amount from 2 wt %, or 2.1 wt %, or 2.2 wt %, or 2.3 wt %, or 2.4 wt %, or 2.5 wt %, or 2.6 wt %, or 2.7 wt %, or 2.8 wt %, or 2.9 wt % to 3 wt %, or 3.1 wt %, or 3.2 wt %, or 3.3 wt %, or 3.4 wt %, or 3.5 wt %, or 3.6 wt %, or 3.7 wt %, or 3.8 wt %, or 3.9 wt %, or 4 wt %, or 4.2 wt %, or 4.4 wt %, or 4.5 wt %.

The artificial sweetener provides a pleasant taste to the aqueous solution without adding meaningful calories or additional flavorings. Nonlimiting examples of suitable artificial sweeteners include xylitol, stevioside, sodium saccharin, and combinations thereof. In an embodiment, the artificial sweetener is xylitol.

### D. Vitamins or Vitamin-Containing Compositions

The aqueous oral composition comprises (D) one or more vitamins or vitamin-containing compositions. The one or more vitamins or vitamin-containing composition(s) is present in an overall amount from 0.1 wt %, or 0.25 wt %, or 0.5 wt %, or 0.75 wt %, or 1 wt %, or 1.25 wt %, or 1.5 wt %, or 1.75 wt %, or 2 wt % to 2.25 wt %, or 2.5 wt %, or 2.75 wt %, or 3 wt %, or 3.25 wt %, or 3.5 wt %, or 3.75 wt %, or 4 wt %. In a further embodiment, the vitamins or vitamin-containing composition(s) is present in an amount from 1.5 wt %, or 1.6 wt %, or 1.7 wt %, or 1.8 wt %, or 1.9 wt %, or 2 wt %, or 2.2 wt % to 2.4 wt %, or 2.5 wt %, or 2.6 wt %, or 2.7 wt %, or 2.8 wt %, or 2.9 wt %, or 3 wt %, or 3.1 wt %, or 3.2 wt %, or 3.3 wt %, or 3.4 wt %, or 3.5 wt %.

In an embodiment, the (D) one or more vitamins or vitamin-containing compositions comprises at least two, or at least three different vitamins or vitamin-containing compositions. In such embodiments, each of the vitamins or vitamin-containing compositions may be present in the aqueous oral composition in the same amount or different amounts. In particular, each vitamin or vitamin-containing composition may be independently present in an amount from 0.1 wt %, or 0.2 wt %, or 0.3 wt %, or 0.4 wt %, or 0.5 wt %, or 0.6 wt %, or 0.7 wt %, or 0.8 wt % to 0.9 wt %, or 1 wt %, or 1.1 wt %, or 1.2 wt %, or 1.3 wt %, or 1.4 wt %, or 1.5 wt %, or 1.6 wt %, or 1.7 wt %, or 1.8 wt %, or 1.9 wt %, or 2 wt %, or 2.5 wt %, or 3 wt %, or 3.5 wt %, or 4 wt %. In a further embodiment, each vitamin or vitamin-containing composition is independently present in an amount from 0.4 wt %, or 0.45 wt %, or 0.5 wt %, or 0.55 wt %, or 0.6 wt %, or 0.65 wt %, or 0.7 wt %, or 0.75 wt %, or 0.8 wt %, or 0.85 wt % to 0.9 wt %, or 0.95 wt %, or 1 wt %, or 1.05 wt %, or 1.1 wt %, or 1.15 wt %, or 1.2 wt %, or 1.25 wt %, or 1.3 wt %, or 1.35 wt %, 1.4 wt %, or 1.45 wt %, or 1.5 wt %, or 1.55 wt %.

Nonlimiting examples of suitable vitamins and vitamin-containing compositions include vitamin C and red algae gel.

In a particular embodiment, the aqueous oral composition comprises vitamin C and red algae gel, wherein:
(i) the vitamin C is present in an amount from 0.1 wt %, or 0.2 wt %, or 0.3 wt %, or 0.4 wt %, or 0.5 wt %, or 0.6 wt %, or 0.7 wt %, or 0.8 wt % to 0.9 wt %, or 1 wt %, or 1.1 wt %, or 1.2 wt %, or 1.3 wt %, or 1.4 wt %, or 1.5 wt %, or 1.6 wt %, or 1.7 wt %, or 1.8 wt %, or 1.9 wt %, or 2 wt %, and preferably from 0.4 wt %, or 0.45 wt %, or 0.5 wt %, or 0.55 wt %, or 0.6 wt %, or 0.65 wt %, or 0.7 wt %, or 0.75 wt %, or 0.8 wt %, or 0.85 wt % to 0.9 wt %, or 0.95 wt %, or 1 wt %, or 1.05 wt %, or 1.1 wt %, or 1.15 wt %, or 1.2 wt %, or 1.25 wt %, or 1.3 wt %, or 1.35 wt %, 1.4 wt %, or 1.45 wt %, or 1.5 wt %, or 1.55 wt %; and/or
(ii) the red algae gel is present in an amount from 0.1 wt %, or 0.2 wt %, or 0.3 wt %, or 0.4 wt %, or 0.5 wt %, or 0.6 wt %, or 0.7 wt %, or 0.8 wt % to 0.9 wt %, or 1 wt %, or 1.1 wt %, or 1.2 wt %, or 1.3 wt %, or 1.4 wt %, or 1.5 wt %, or 1.6 wt %, or 1.7 wt %, or 1.8 wt %, or 1.9 wt %, or 2 wt %, and preferably from 0.4 wt %, or 0.45 wt %, or 0.5 wt %, or 0.55 wt %, or 0.6 wt %, or 0.65 wt %, or 0.7 wt %, or 0.75 wt %, or 0.8 wt %, or 0.85 wt % to 0.9 wt %, or 0.95 wt %, or 1 wt %, or 1.05 wt %, or 1.1 wt %, or 1.15 wt %, or 1.2 wt %, or 1.25 wt %, or 1.3 wt %, or 1.35 wt %, 1.4 wt %, or 1.45 wt %, or 1.5 wt %, or 1.55 wt %; and/or
(iii) the total amount of the vitamin C and red algae gel combined is from 0.1 wt %, or 0.25 wt %, or 0.5 wt %, or 0.75 wt %, or 1 wt %, or 1.25 wt %, or 1.5 wt %, or 1.75 wt %, or 2 wt % to 2.25 wt %, or 2.5 wt %, or 2.75 wt %, or 3 wt %, or 3.25 wt %, or 3.5 wt %, or 3.75 wt %, or 4 wt %, and preferably from 1.5 wt %, or 1.6 wt %, or 1.7 wt %, or 1.8 wt %, or 1.9 wt %, or 2 w1%, or 2.2 wt % to 2.4 wt %, or 2.5 wt %, or 2.6 wt %, or 2.7 wt %, or 2.8 wt %, or 2.9 wt %, or 3 wt %, or 3.1 wt %, or 3.2 wt %, or 3.3 wt %, or 3.4 wt %, or 3.5 wt %.

### E. Grape Seed Extract

The aqueous oral composition comprises (E) grape seed extract. The grape seed extract is present in an amount from 0.1 wt %, or 0.2 wt %, or 0.3 wt %, or 0.4 wt % to 0.5 wt %, or 0.6 wt %, or 0.7 wt %, or 0.8 wt %, or 0.9 wt %, or 1 wt %. In a further embodiment, the grape seed extract is present in an amount from 0.3 wt %, or 0.35 wt %, or 0.4 wt %, or 0.45 wt % to 0.5 wt %, or 0.55 wt %, or 0.6 wt %, or 0.65 wt %, or 0.7 wt %, or 0.75 wt %.

Grape seed extract is a known oral protector.

### F. Miswak Powder

The aqueous oral composition comprises (F) miswak powder. The miswak powder is present in an amount from 0.1 wt %, or 0.2 wt %, or 0.3 wt %, or 0.4 wt % to 0.5 wt %, or 0.6 wt %, or 0.7 wt %, or 0.8 wt %, or 0.9 wt %, or 1 wt %, or 2 wt %, or 3 wt %, or 4 wt %, or 5 wt %, or 10 wt %, or 20 wt %. In a further embodiment, the miswak powder is present in an amount from 0.3 wt %, or 0.32 wt %, or 0.34 wt %, or 0.36 wt %, or 0.38 wt %, or 0.4 wt %, or 0.42 wt %, or 0.44 wt %, or 0.46 wt %, or 0.48 wt % to 0.5 wt %, or 0.52 wt %, or 0.54 wt %, or 0.56 wt %, or 0.58 wt %, or 0.6 wt %, or 0.62 wt %, or 0.64 wt %, or 0.66 wt %, or 0.68 wt %, or 0.7 wt %.

Miswak powder is a known teeth cleaning agent.

### G. Additional Components

The aqueous oral composition may comprise additional components which contribute to the effectiveness, taste, and/or other properties of the aqueous oral composition. Nonlimting examples of additional components include preservatives, solvents, emulsifiers, natural oils, surfactants, and combinations thereof.

In an embodiment, the aqueous oral composition comprises a preservative. Nonlimiting examples of suitable preservatives include benzyl alcohol and cetylpyrdium chloride. In an embodiment, the preservative is present in the aqueous oral composition in an amount from 0.1 wt %, or 0.2 wt %, or 0.3 wt %, or 0.4 wt %, or 0.5 wt % to 0.6 wt %, or 0.7 wt %, or 0.8 wt %, or 0.9 wt %, or 1 wt %. In a further embodiment, the preservative is present in an amount from 0.3 wt %, or 0.32 wt %, or 0.34 wt %, or 0.36 wt %, or 0.38 wt %, or 0.4 wt %, or 0.42 wt %, or 0.44 wt %, or 0.46 wt %, or 0.48 wt %, or 0.5 wt %, or 0.52 wt %, or 0.54 wt %, or 0.56 wt %, or 0.58 wt % to 0.6 wt %, or 0.62 wt %, or 0.64 wt %, or 0.66 wt %, or 0.68 wt %, or 0.7 wt %, or 0.72 wt %, or 0.74 wt %, or 0.76 wt %, or 0.78 wt %, or 0.8 wt %.

In an embodiment, the aqueous oral composition comprises a solvent and/or emulsifier. In some embodiments, a single component may act as a solvent and emulsifier. A nonlimiting example of a suitable solvent and emulsifier is triethylene glycol. In an embodiment, the solvent and/or emulsifier is present in the aqueous oral solution in an amount from 0.1 wt %, or 0.15 wt %, or 0.2 wt % to 0.25 wt %, or 0.3 wt %. In a further embodiment, the solvent and/or emulsifier is present in an amount from 0.15 wt %, or 0.16 wt %, or 0.17 wt %, or 0.18 wt %, or 0.19 wt %, or 0.2 wt %, or 0.21 wt %, or 0.22 wt %, or 0.23 wt %, or 0.24 wt % to 0.25 wt %, or 0.26 wt %, or 0.27 wt %, or 0.28 wt %, or 0.29 wt %, or 0.3 wt %.

In an embodiment, the aqueous oral composition comprises a natural oil. A nonlimiting example of a suitable natural oil is fractionated coconut oil. In an embodiment, the natural oil is present in an amount from 0.1 wt %, or 0.2 wt %, or 0.3 wt %, or 0.4 wt %, or 0.5 wt % to 0.6 wt %, or 0.7 wt %, or 0.8 wt %, or 0.9 wt %, or 1 wt %. In a further embodiment, the natural oil is present in an amount from 0.4 wt %, or 0.42 wt %, or 0.44 wt %, or 0.46 wt %, or 0.48 wt %, or 0.5 wt %, or 0.52 wt %, or 0.54 wt %, or 0.56 wt %, or 0.58 wt % to 0.6 wt %, or 0.62 wt %, or 0.64 wt %, or 0.66 wt %, or 0.68 wt %, or 0.7 wt %, or 0.72 wt %, or 0.74 wt %, or 0.76 wt %.

In an embodiment, the aqueous oral composition comprises a surfactant. Nonlimiting examples of suitable surfactants include glycolipids. In an embodiment, the surfactant is present in an amount from 0.1 wt %, or 0.2 wt %, or 0.3 wt %, or 0.4 wt %, or 0.5 wt % to 0.6 wt %, or 0.7 wt %, or 0.8 wt %, or 0.9 wt %, or 1 wt %. In a further embodiment, the surfactant is present in an amount from 0.4 wt %, or 0.42 wt %, or 0.44 wt %, or 0.46 wt %, or 0.48 wt %, or 0.5 wt %, or 0.52 wt %, or 0.54 wt %, or 0.56 wt %, or 0.58 wt % to 0.6 wt %, or 0.62 wt %, or 0.64 wt %, or 0.66 wt %, or 0.68 wt %, or 0.7 wt %, or 0.72 wt %, or 0.74 wt %, or 0.76 wt %.

### H. Water

The present disclosure provides an aqueous oral composition. The aqueous oral composition therefore comprises a majority amount water. In an embodiment, the water is present in an amount from 40 wt %, or 45 wt %, or 50 wt % to 55 wt %, or 60 wt %, or 65 wt %, or 70 wt %, or 75 wt %. In a further embodiment, the water is present in an amount from 45 wt %, or 46 wt %, or 47 wt %, or 48 wt %, or 49 wt %, or 50 wt %, or 51 wt %, or 52 wt %, or 53 wt %, or 54 wt % to 55 wt %, or 56 wt %, or 57 wt %, or 58 wt %, or 59 wt %, or 60 wt %, or 61 wt %, or 62 wt %, or 63 wt %, or 64 wt %, or 65 wt %.

### Aqueous Oral Composition

In an embodiment, an aqueous oral composition comprises, consists essentially of, or consists of water and (A) an alcohol, (B) sodium bicarbonate, (C) an artificial sweetener, (D) one or more vitamins or vitamin-containing compositions, (E) grape seed extract, and (F) miswak powder.

In an embodiment, an aqueous oral composition comprises, consists essentially of, or consists of from 40 wt %, or 45 wt %, or 50 wt % to 55 wt %, or 60 wt %, or 65 wt %, or 70 wt %, or 75 wt % water and
(A) from 5 wt %, or 10 wt %, or 15 wt % to 20 wt %, or 25 wt % of an alcohol according to any embodiment or combination or embodiments provided herein,
(B) from 1 wt %, or 2 wt %, or 3 wt %, or 4 wt %, or 5 wt %, or 6 wt % to 7 wt %, or 8 wt %, or 9 wt %, or 10 wt %, or 11 wt %, or 12 wt % sodium bicarbonate,
(C) from 1 wt %, or 1.5 wt %, or 2 wt %, or 2.5 wt % to 3 wt %, or 3.5 wt %, or 4 wt %, or 4.5 wt %, or 5 wt % of an artificial sweetener according to any embodiment or combination or embodiments provided herein,
(D) from 0.1 wt %, or 0.25 wt %, or 0.5 wt %, or 0.75 wt %, or 1 wt %, or 1.25 wt %, or 1.5 wt %, or 1.75 wt %, or 2 wt % to 2.25 wt %, or 2.5 wt %, or 2.75 wt %, or 3 wt %, or 3.25 wt %, or 3.5 wt %, or 3.75 wt %, or 4 wt % of one or more vitamins or vitamin-containing compositions according to any embodiment or combination or embodiments provided herein,
(E) from 0.1 wt %, or 0.2 wt %, or 0.3 wt %, or 0.4 wt % to 0.5 wt %, or 0.6 wt %, or 0.7 wt %, or 0.8 wt %, or 0.9 wt %, or 1 wt % grape seed extract, and
(F) from 0.1 wt %, or 0.2 wt %, or 0.3 wt %, or 0.4 wt % to 0.5 wt %, or 0.6 wt %, or 0.7 wt %, or 0.8 wt %, or 0.9 wt %, or 1 wt % miswak powder.

In an embodiment, the one or more vitamins or vitamin-containing compositions comprises two or more vitamins or vitamin-containing compositions, wherein each vitamin or vitamin-containing compositions is each independently present in an amount from 0.1 wt %, or 0.2 wt %, or 0.3 wt %, or 0.4 wt %, or 0.5 wt %, or 0.6 wt %, or 0.7 wt %, or 0.8 wt % to 0.9 wt %, or 1 wt %, or 1.1 wt %, or 1.2 wt %, or 1.3 wt %, or 1.4 wt %, or 1.5 wt %, or 1.6 wt %, or 1.7 wt %, or 1.8 wt %, or 1.9 wt %, or 2 wt %, or 2.5 wt %, or 3 wt %, or 3.5 wt % or 4 wt %.

In an embodiment, an aqueous oral composition comprises, consists essentially of, or consists of water and (A) an alcohol, (B) sodium bicarbonate, (C) an artificial sweetener, (D) one or more vitamins or vitamin-containing compositions, (E) grape seed extract, (F) miswak powder, and (G) one or more additional components selected from the group consisting of preservatives, solvents, emulsifiers, natural oils, surfactants and combinations thereof.

In an embodiment, an aqueous oral composition comprises, consists essentially of, or consists of from 40 wt %, or 45 wt %, or 50 wt % to 55 wt %, or 60 wt %, or 65 wt %, or 70 wt %, or 75 wt % water and
(A) from 5 wt %, or 10 wt %, or 15 wt % to 20 wt %, or 25 wt % of an alcohol according to any embodiment or combination of embodiments provided herein,
(B) from 1 wt %, or 2 wt %, or 3 wt %, or 4 wt %, or 5 wt %, or 6 wt % to 7 wt %, or 8 wt %, or 9 wt %, or 10 wt %, or 11 wt %, or 12 wt % sodium bicarbonate,
(C) from 1 wt %, or 1.5 wt %, or 2 wt %, or 2.5 wt % to 3 wt %, or 3.5 wt %, or 4 wt %, or 4.5 wt %, or 5 wt % of an artificial sweetener according to any embodiment or combination of embodiments provided herein,
(D) from 0.1 wt %, or 0.25 wt %, or 0.5 wt %, or 0.75 wt %, or 1 wt %, or 1.25 wt %, or 1.5 wt %, or 1.75 wt %, or 2 wt % to 2.25 wt %, or 2.5 wt %, or 2.75 wt %, or 3 wt %, or 3.25 wt %, or 3.5 wt %, or 3.75 wt %, or 4 wt % of one or more vitamins or vitamin-containing compositions according to any embodiment or combination of embodiments provided herein,
(E) from 0.1 wt %, or 0.2 wt %, or 0.3 wt %, or 0.4 wt % to 0.5 wt %, or 0.6 wt %, or 0.7 wt %, or 0.8 wt %, or 0.9 wt %, or 1 wt % grape seed extract,
(F) from 0.1 wt %, or 0.2 wt %, or 0.3 wt %, or 0.4 wt % to 0.5 wt %, or 0.6 wt %, or 0.7 wt %, or 0.8 wt %, or 0.9 wt %, or 1 wt % miswak powder, and
(G) one or more additional components selected from the group consisting of:
   (i) from 0.1 wt %, or 0.2 wt %, or 0.3 wt %, or 0.4 wt %, or 0.5 wt % to 0.6 wt %, or 0.7 wt %, or 0.8 wt %, or 0.9 wt %, or 1 wt % of a preservative,
   (ii) from 0.1 wt %, or 0.15 wt %, or 0.2 wt % to 0.25 wt %, or 0.3 wt % of a solvent and/or emulsifier,
   (iii) from 0.1 wt %, or 0.2 wt %, or 0.3 wt %, or 0.4 wt %, or 0.5 wt % to 0.6 wt %, or 0.7 wt %, or 0.8 wt %, or 0.9 wt %, or 1 wt % of a natural oil,
   (iv) from 0.1 wt %, or 0.2 wt %, or 0.3 wt %, or 0.4 wt %, or 0.5 wt % to 0.6 wt %, or 0.7 wt %, or 0.8 wt %, or 0.9 wt %, or 1 wt % of a surfactant, and
   (v) combinations thereof.

In an embodiment, the one or more vitamins or vitamin-containing compositions comprises two or more vitamins or vitamin-containing compositions, wherein each vitamin or vitamin-containing compositions is each independently present in an amount from 0.1 wt %, or 0.2 wt %, or 0.3 wt %, or 0.4 wt %, or 0.5 wt %, or 0.6 wt %, or 0.7 wt %, or 0.8 wt % to 0.9 wt %, or 1 wt %, or 1.1 wt %, or 1.2 wt %, or 1.3 wt %, or 1.4 wt %, or 1.5 wt %, or 1.6 wt %, or 1.7 wt %, or 1.8 wt %, or 1.9 wt %, or 2 wt % or 2.5 wt %, or 3 wt %, or 3.5 wt % or 4 wt %.

In an embodiment, an aqueous oral composition comprises, consists essentially of, or consists of water and (A) an alcohol selected from the group consisting of glycerin, sorbitol and combinations thereof, (B) sodium bicarbonate, (C) an artificial sweetener selected from the group consisting of xylitol, stevioside, sodium saccharin, and combinations thereof, (D) one or more vitamins or vitamin-containing compositions selected from the group consisting of vitamin C, red algae extract, and combinations thereof, (E) grape seed extract, (F) miswak powder, and (G) one or more additional components selected from the group consisting of preservatives, solvents, emulsifiers, natural oils, surfactants and combinations thereof.

In an embodiment, an aqueous oral composition comprises, consists essentially of, or consists of from 40 wt %, or 45 wt %, or 50 wt % to 55 wt %, or 60 wt %, or 65 wt %, or 70 wt %, or 75 wt % water and
(A) from 5 wt %, or 10 wt %, or 15 wt % to 20 wt %, or 25 wt % of an alcohol selected from the group consisting of glycerin, sorbitol and combinations thereof,
(B) from 1 wt %, or 2 wt %, or 3 wt %, or 4 wt %, or 5 wt %, or 6 wt % to 7 wt %, or 8 wt %, or 9 wt %, or 10 wt %, or 11 wt %, or 12 wt % sodium bicarbonate,
(C) from 1 wt %, or 1.5 wt %, or 2 wt %, or 2.5 wt % to 3 wt %, or 3.5 wt %, or 4 wt %, or 4.5 wt %, or 5 wt % of an artificial sweetener selected from the group consisting of xylitol, stevioside, sodium saccharin, and combinations thereof,
(D) from 0.1 wt %, or 0.25 wt %, or 0.5 wt %, or 0.75 wt %, or 1 wt %, or 1.25 wt %, or 1.5 wt %, or 1.75 wt %, or 2 wt % to 2.25 wt %, or 2.5 wt %, or 2.75 wt %, or 3 wt %, or 3.25 wt %, or 3.5 wt %, or 3.75 wt %, or 4 wt % of one or more vitamins or vitamin-containing compositions selected from the group consisting of vitamin C, red algae gel and combinations thereof,
(E) from 0.1 wt %, or 0.2 wt %, or 0.3 wt %, or 0.4 wt % to 0.5 wt %, or 0.6 wt %, or 0.7 wt %, or 0.8 wt %, or 0.9 wt %, or 1 wt % grape seed extract,
(F) from 0.1 wt %, or 0.2 wt %, or 0.3 wt %, or 0.4 wt % to 0.5 wt %, or 0.6 wt %, or 0.7 wt %, or 0.8 wt %, or 0.9 wt %, or 1 wt % miswak powder, and
(G) one or additional components selected from the group consisting of:
   (i) from 0.1 wt %, or 0.2 wt %, or 0.3 wt %, or 0.4 wt %, or 0.5 wt % to 0.6 wt %, or 0.7 wt %, or 0.8 wt %, or 0.9 wt %, or 1 wt % of a preservative selected from the group consisting of benzyl alcohol, cetylpyrdium chloride, and combinations thereof,
   (ii) from 0.1 wt %, or 0.15 wt %, or 0.2 wt % to 0.25 wt %, or 0.3 wt % of a solvent and/or emulsifier which is triethylene glycol,
   (iii) from 0.1 wt %, or 0.2 wt %, or 0.3 wt %, or 0.4 wt %, or 0.5 wt % to 0.6 wt %, or 0.7 wt %, or 0.8 wt %, or 0.9 wt %, or 1 wt % of a natural oil which is fractionated coconut oil,
   (iv) from 0.1 wt %, or 0.2 wt %, or 0.3 wt %, or 0.4 wt %, or 0.5 wt % to 0.6 wt %, or 0.7 wt %, or 0.8 wt %, or 0.9 wt %, or 1 wt % of a surfactant comprising glycolipids, and
   (v) combinations thereof.

In an embodiment, the one or more vitamins or vitamin-containing compositions comprises vitamin C and red algae gel, each of which is independently present in an amount from 0.1 wt %, or 0.2 wt %, or 0.3 wt %, or 0.4 wt %, or 0.5 wt %, or 0.6 wt %, or 0.7 wt %, or 0.8 wt % to 0.9 wt %, or 1 wt %, or 1.1 wt %, or 1.2 wt %, or 1.3 wt %, or 1.4 wt %, or 1.5 wt %, or 1.6 wt %, or 1.7 wt %, or 1.8 wt %, or 1.9 wt %, or 2 wt %, or 2.5 wt %, or 3 wt %, or 3.5 wt %, or 4wt %.

In an embodiment, an aqueous oral composition comprises, consists essentially of, or consists of water and (A) an alcohol selected from the group consisting of glycerin, sorbitol and combinations thereof, (B) sodium bicarbonate, (C) an artificial sweetener selected from the group consisting of xylitol, stevioside, sodium saccharin, and combinations thereof, (D) one or more vitamins or vitamin-containing compositions selected from the group consisting of vitamin C, red algae extract, and combinations thereof, (E) grape seed extract, (F) miswak powder, and (G) the additional components of (i) a preservative, (ii) a solvent/emulsifier, (iii) a natural oil, and (iv) a surfactant.

In an embodiment, an aqueous oral composition comprises, consists essentially of, or consists of from 40 wt %, or 45 wt %, or 50 wt % to 55 wt %, or 60 wt %, or 65 wt %, or 70 wt %, or 75 wt % water and
(A) from 5 wt %, or 10 wt %, or 15 wt % to 20 wt %, or 25 wt % of an alcohol selected from the group consisting of glycerin, sorbitol and combinations thereof,
(B) from 1 wt %, or 2 wt %, or 3 wt %, or 4 wt %, or 5 wt %, or 6 wt % to 7 wt %, or 8 wt %, or 9 wt %, or 10 wt %, or 11 wt %, or 12 wt % sodium bicarbonate,
(C) from 1 wt %, or 1.5 wt %, or 2 wt %, or 2.5 wt % to 3 wt %, or 3.5 wt %, or 4 wt %, or 4.5 wt %, or 5 wt % of an artificial sweetener selected from the group consisting of xylitol, stevioside, sodium saccharin, and combinations thereof,
(D) from 0.1 wt %, or 0.25 wt %, or 0.5 wt %, or 0.75 wt %, or 1 wt %, or 1.25 wt %, or 1.5 wt %, or 1.75 wt %, or 2 wt % to 2.25 wt %, or 2.5 wt %, or 2.75 wt %, or 3 wt %, or 3.25 wt %, or 3.5 wt %, or 3.75 wt %, or 4 wt % of one or more vitamins or vitamin-containing compositions selected from the group consisting of vitamin C, red algae gel and combinations thereof,
(E) from 0.1 wt %, or 0.2 wt %, or 0.3 wt %, or 0.4 wt % to 0.5 wt %, or 0.6 wt %, or 0.7 wt %, or 0.8 wt %, or 0.9 wt %, or 1 wt % grape seed extract,
(F) from 0.1 wt %, or 0.2 wt %, or 0.3 wt %, or 0.4 wt % to 0.5 wt %, or 0.6 wt %, or 0.7 wt %, or 0.8 wt %, or 0.9 wt %, or 1 wt % miswak powder, and
(G) the additional components:
   (i) from 0.1 wt %, or 0.2 wt %, or 0.3 wt %, or 0.4 wt %, or 0.5 wt % to 0.6 wt %, or 0.7 wt %, or 0.8 wt %, or 0.9 wt %, or 1 wt % of a preservative selected from the group consisting of benzyl alcohol, cetylpyrdium chloride, and combinations thereof,
   (ii) from 0.1 wt %, or 0.15 wt %, or 0.2 wt % to 0.25 wt %, or 0.3 wt % of a solvent and/or emulsifier which is triethylene glycol,
   (iii) from 0.1 wt %, or 0.2 wt %, or 0.3 wt %, or 0.4 wt %, or 0.5 wt % to 0.6 wt %, or 0.7 wt %, or 0.8 wt %, or 0.9 wt %, or 1 wt % of a natural oil which is fractionated coconut oil, and
   (iv) from 0.1 wt %, or 0.2 wt %, or 0.3 wt %, or 0.4 wt %, or 0.5 wt % to 0.6 wt %, or 0.7 wt %, or 0.8 wt %, or 0.9 wt %, or 1 wt % of a surfactant comprising glycolipids.

In a particular embodiment, the aqueous oral composition comprises, consists essentially of, or consists of from 45 wt %, or 46 wt %, or 47 wt %, or 48 wt %, or 49 wt %, or 50 wt %, or 51 wt %, or 52 wt %, or 53 wt %, or 54 wt % to 55 wt %, or 56 wt %, or 57 wt %, or 58 wt %, or 59 wt %, or 60 wt %, or 61 wt %, or 62 wt %, or 63 wt %, or 64 wt %, or 65 wt % water and
(A) from 10 wt %, or 12 wt %, or 14 wt %, or 16 wt %, or 18 wt % to 20 wt %, or 22 wt %, or 24 wt %, or 25 wt % of an alcohol selected from the group consisting of glycerin, sorbitol and combinations thereof,
(B) from 3 wt %, or 3.25 wt %, or 3.5 wt %, or 3.75 wt %, or 4 wt %, or 4.25 wt %, or 4.5 wt %, or 4.75 wt %, or 5 wt %, or 5.25 wt %, or 5.5 wt %, or 5.75 wt %, or 6 wt %, or 6.25 wt %, or 6.5 wt %, or 6.75 wt % to 7 wt %, or 7.25 wt %, or 7.5 wt %, or 7.75 wt %, or 8 wt %, or 8.25 wt %, or 8.5 wt %, or 8.75 wt %, or 9 wt %, or 9.5 wt %, or 10 wt % sodium bicarbonate,
(C) from 2 wt %, or 2.1 wt %, or 2.2 wt %, or 2.3 wt %, or 2.4 wt %, or 2.5 wt %, or 2.6 wt %, or 2.7 wt %, or 2.8 wt %, or 2.9 wt % to 3 wt %, or 3.1 wt %, or 3.2 wt %, or 3.3 wt %, or 3.4 wt %, or 3.5 wt %, or 3.6 wt %, or 3.7 wt %, or 3.8 wt %, or 3.9 wt %, or 4 wt %, or 4.2 wt %, or 4.4 wt %, or 4.5 wt % of an artificial sweetener selected from the group consisting of xylitol, stevioside, sodium saccharin, and combinations thereof,
(D) from 1.5 wt %, or 1.6 wt %, or 1.7 wt %, or 1.8 wt %, or 1.9 wt %, or 2 wt %, or 2.2 wt % to 2.4 wt %, or 2.5 wt %, or 2.6 wt %, or 2.7 wt %, or 2.8 wt %, or 2.9 wt %, or 3 wt %, or 3.1 wt %, or 3.2 wt %, or 3.3 wt %, or 3.4 wt %, or 3.5 wt % of one or more vitamins or vitamin-containing compositions selected from the group consisting of vitamin C, red algae gel and combinations thereof,
(E) from 0.3 wt %, or 0.35 wt %, or 0.4 wt %, or 0.45 wt % to 0.5 wt %, or 0.55 wt %, or 0.6 wt %, or 0.65 wt %, or 0.7 wt %, or 0.75 wt % grape seed extract,
(F) from 0.3 wt %, or 0.32 wt %, or 0.34 wt %, or 0.36 wt %, or 0.38 wt %, or 0.4 wt %, or 0.42 wt %, or 0.44 wt %, or 0.46 wt %, or 0.48 wt % to 0.5 wt %, or 0.52 wt %, or 0.54 wt %, or 0.56 wt %, or 0.58 wt %, or 0.6 wt %, or 0.62 wt %, or 0.64 wt %, or 0.66 wt %, or 0.68 wt %, or 0.7 wt % miswak powder, and
(G) the additional components:
   (i) from 0.3 wt %, or 0.32 wt %, or 0.34 wt %, or 0.36 wt %, or 0.38 wt %, or 0.4 wt %, or 0.42 wt %, or 0.44 wt %, or 0.46 wt %, or 0.48 wt %, or 0.5 wt %, or 0.52 wt %, or 0.54 wt %, or 0.56 wt %, or 0.58 wt % to 0.6 wt %, or 0.62 wt %, or 0.64 wt %, or 0.66 wt %, or 0.68 wt %, or 0.7 wt %, or 0.72 wt %, or 0.74 wt %, or 0.76 wt %, or 0.78 wt %, or 0.8 wt % of a preservative selected from the group consisting of benzyl alcohol, cetylpyrdium chloride, and combinations thereof,
   (ii) from 0.15 wt %, or 0.16 wt %, or 0.17 wt %, or 0.18 wt %, or 0.19 wt %, or 0.2 wt %, or 0.21 wt %, or 0.22 wt %, or 0.23 wt %, or 0.24 wt % to 0.25 wt %, or 0.26 wt %, or 0.27 wt %, or 0.28 wt %, or 0.29 wt %, or 0.3 wt % of a solvent and/or emulsifier which is triethylene glycol,
   (iii) from 0.4 wt %, or 0.42 wt %, or 0.44 wt %, or 0.46 wt %, or 0.48 wt %, or 0.5 wt %, or 0.52 wt %, or 0.54 wt %, or 0.56 wt %, or 0.58 wt % to 0.6 wt %, or 0.62 wt %, or 0.64 wt %, or 0.66 wt %, or 0.68 wt %, or 0.7 wt %, or 0.72 wt %, or 0.74 wt %, or 0.76 wt % of a natural oil which is fractionated coconut oil, and
   (iv) from 0.4 wt %, or 0.42 wt %, or 0.44 wt %, or 0.46 wt %, or 0.48 wt %, or 0.5 wt %, or 0.52 wt %, or 0.54 wt %, or 0.56 wt %, or 0.58 wt % to 0.6 wt %, or 0.62 wt %, or 0.64 wt %, or 0.66 wt %, or 0.68 wt %, or 0.7 wt %, or 0.72 wt %, or 0.74 wt %, or 0.76 wt % of a surfactant comprising glycolipids.

In a further embodiment, the aqueous oral composition comprises, consists essentially of, or consists of from 65 wt % to 75 wt % of water and
(A) from 15 wt % to 25 wt % of an alcohol selected from the group consisting of glycerin, sorbitol and combinations thereof,
(B) from 1 wt % to 5 wt % of sodium bicarbonate,
(C) from 1 wt % to 5 wt % of an artificial sweetener selected from the group consisting of xylitol, stevioside, sodium saccharin, and combinations thereof,
(D) from 0.5 wt % to 0.9 wt % of vitamin C or a vitamin C-containing composition,
(E) from 0.01 wt. % to 0.09 wt % of red algae extract,
(F) from 0.1 wt % to 0.5 wt % of grape seed extract,
(G) from 0.1 wt % to 0.5 wt % of miswak powder, and
(G) the additional components:
   (i) from 0.1 wt % to 0.9 wt % of a preservative selected from the group consisting of benzyl alcohol, cetylpyrdium chloride, and combinations thereof,
   (ii) from 0.01 wt % to 0.09 wt % of a solvent and/or emulsifier which is triethylene glycol,
   (iii) from 0.1 wt % to 0.9 wt % of a natural oil which is fractionated coconut oil, for example an oil containing caprylic/capric triglyceride, and
   (iv) from 0.1 wt % to 0.5 wt % of a surfactant comprising glycolipids.

The present disclosure provides a method of using an aqueous oral composition. The aqueous oral composition is in accordance with any embodiment or combination of embodiments provided hereon. In an embodiment, the aqueous oral composition is provided as an oral spray, a rinse, a liquid applied via applicator (e.g., a brush, swab, bristles, etc.), and combinations thereof.

In an embodiment, the aqueous oral composition is provided as an oral spray. In such an embodiment, the method of using the aqueous oral composition comprises spraying one or more sprays in the oral cavity, and preferably spraying one or more sprays on a user's teeth. In a particular embodiment, the method of spraying one or more sprays in the oral cavity, and preferably on the user's teeth, comprises spraying a first spray at a first of a left or right side of an oral cavity, spraying a second spray in the middle of the oral cavity, and spraying a third spray at the other of the left or right side of the oral cavity. In an embodiment, the method further includes spraying a fourth spray at the center of the oral cavity. While the sprays are described as “first,” “second,” “third,” “fourth,” etc., it will be appreciated that the sprays may occur in any order.

In an embodiment, a user completes a first application prior to drinking a teeth-staining beverage, such as wine, coffee, or other dark pigmented food or beverage. In an embodiment, a user evaluates the need for a subsequent application after finishing each glass of the teeth-staining beverage or consuming a substantial amount of food.

In another embodiment, the aqueous oral composition is provided as a rinse. In such an embodiment, the method of using the aqueous oral composition comprises swishing a volume of the aqueous oral composition in the oral cavity for a duration of time and spitting the aqueous oral composition out. In an embodiment, a user evaluates the need for a subsequent application after finishing each glass of the teeth-staining beverage or consuming a substantial amount of food.

In another embodiment, the aqueous oral composition is provided as a liquid to be applied with an applicator. In such an embodiment, the method of using the aqueous oral composition comprises applying, with the applicator, an amount of aqueous oral composition directly to the surfaces of the teeth. In an embodiment, a user evaluates the need for a subsequent application after finishing each glass of the teeth-staining beverage or consuming a substantial amount food.

### EXAMPLES

The following examples are to be considered as merely illustrative and are not intended to limit the scope of this invention.

### Example 1

A composition was prepared using the following ingredients in the listed ranges:

TABLE 1
Composition ingredients.

| | Min. % w/w | Max. % w/w |
|---|---|---|
| Ingredient | of INCI | of INCI |
| Water | 65.0 | 75.0 |
| Glycerin | 15.0 | 25.0 |
| Sodium bicarbonate | 3.0 | 6.0 |
| Xylitol | 1.0 | 5.0 |
| Benzyl alcohol | 0.1 | 0.9 |
| Caprylic/capric triglyceride | 0.1 | 0.9 |
| (coconut oil) | | |
| Salvadora persica bark/root | 0.1 | 0.5 |
| extract (miswak powder) | | |
| Ascorbic acid (Vitamin C) | 0.5 | 0.9 |
| Glycolipids | 0.1 | 0.5 |
| Vitis vinifera (grape) seed | 0.1 | 0.5 |
| extract | | |
| Ahnfeltiopsis concinna | 0.01 | 0.09 |
| (red algae) extract | | |
| Triethylene glycol | 0.01 | 0.09 |

All water-soluble ingredients were mixed in a cold process. Then, all oil-soluble ingredients were added, with coconut oil being added to the bulk last. The composition was mixed in a GMP standard mixing vessel with high-shear homogenization. Stability testing was conducted on the resulting composition and occurred at room temperature, at 30° C., at 40° C., and in a refrigerator at 4° C. The composition was found to be stable at all temperatures tested.

It is specifically intended that the present disclosure not be limited to the embodiments and illustrations contained herein, but include modified forms of those embodiments including portions of the embodiments and combinations of elements of different embodiments as come within the scope of the following claims.

We claim:

1. An aqueous oral composition comprising (A) an alcohol, (B) sodium bicarbonate, (C) an artificial sweetener, (D) one or more vitamins or vitamin-containing compositions, (E) grape seed extract, and (F) miswak powder.

2. The aqueous oral composition of claim 1, wherein the alcohol is present in an amount from 5 wt % to 25 wt %.

3. The aqueous oral composition of claim 1, wherein the alcohol is selected from the group consisting of glycerin, sorbitol, and combinations thereof.

4. The aqueous oral composition of claim 1, wherein the sodium bicarbonate is present in an amount from 1 wt % to 12 wt %.

5. The aqueous oral composition of claim 1, wherein the artificial sweetener is present in an amount from 1 wt % to 5 wt %.

6. The aqueous oral composition of claim 1, wherein the artificial sweetener is selected from the group consisting of xylitol, stevioside, sodium saccharin, and combinations thereof.

7. The aqueous oral composition of claim 1, wherein the one or more vitamins or vitamin-containing compositions is present in an amount from 0.1 wt % to 4 wt %.

8. The aqueous oral composition of claim 1, wherein the one or more vitamins or vitamin-containing compositions is selected from the group consisting of vitamin C, red algae gel, and combinations thereof.

9. The aqueous oral composition of claim 8, wherein the one or more vitamins or vitamin-containing compositions comprises vitamin C and red algae gel.

10. The aqueous oral composition of claim 9, wherein the vitamin C and red algae gel are independently present in an amount from 0.1 wt % to 2 wt %.

11. The aqueous oral composition of claim 1, wherein the grape seed extract is present in an amount from 0.1 wt % to 1 wt %.

12. The aqueous oral composition of claim 1, wherein the miswak powder is present in an amount from 0.1 wt % to 1 wt %.

13. The aqueous oral composition of claim 1, further comprising an additional component selected from the group consisting of preservatives, solvents, emulsifiers, natural oils, surfactants and combinations thereof.

14. The aqueous oral composition of claim 13 further comprising a preservative selected from the group consisting of benzyl alcohol, cetylpyrdium chloride, and combinations thereof.

15. The aqueous oral composition of claim 13 further comprising a solvent/emulsifier which is triethylene glycol.

16. The aqueous oral composition of claim 13 further comprising a natural oil which is fractionated coconut oil.

17. The aqueous oral composition of claim 13 further comprising a surfactant comprising glycolipids.

18. The aqueous oral composition of claim 1 provided as a spray, rinse, or liquid to be applied with an applicator.

19. A method of using the aqueous oral composition of claim 1, the method comprising spraying the aqueous oral composition in an oral cavity.

20. The method of claim 19, wherein the step of spraying the aqueous oral composition in an oral cavity comprises spraying the aqueous oral composition on a user's teeth.
